(19) United States
(12) Patent Application Publication
(10) Pub. No.: US 2017/0014281 A1
(43) Pub. Date: Jan. 19, 2017

(54) THREE-DIMENSIONAL NONWOVEN FABRIC WITH PORE STRUCTURE

(71) Applicant: XIAMEN YANJAN NEW MATERIAL CO., LTD, Xiamen, Fujian (CN)

(72) Inventors: Jihua XIE, Xiamen (CN); Jiquan XIE, Xiamen (CN)

(21) Appl. No.: 15/124,354

(22) PCT Filed: May 29, 2014
(86) PCT No.: PCT/CN2014/078729

§ 371 Date: Sep. 8, 2016

(30) Foreign Application Priority Data
Mar. 26, 2014 (CN) 201420140109.0

(51) Int. Cl. A61F 13/511; D04H 13/00; A61F 13/512

(52) U.S. Cl. CPC A61F 13/51104; A61F 13/512; D04H 13/00

(57) ABSTRACT

A three-dimensional nonwoven fabric with a pore structure includes raised regions, recessed regions, and the pore structure. Fiber densities from the raised regions to the recessed regions are gradient. That is, the fiber density of the edges transiting from the raised regions to the recessed regions is higher than that of the tops of the raised regions. Pressure resistance performance of the high-density fiber regions keeps the three-dimensional nonwoven fabric significantly uneven, and quickens liquid permeation and reduces residues, and the three-dimensional nonwoven fabric has a high permeating speed and is dry and comfortable.

### BACKGROUND OF THE INVENTION

1. Field of the Invention

The present invention relates to a nonwoven fabric, and more particularly to a three-dimensional nonwoven fabric having raised regions, recessed regions, and a pore structure.

2. Description of the Prior Art

These days, nonwoven fabrics are widely used to sanitary absorbent products, such as sanitary napkins, diapers and the like, cleaning supplies, masks, medical supplies, and so on. In order to improve the comfort of the nonwoven fabrics to contact with the skin, a nonwoven fabric having a concave-convex structure is developed. The surface of the nonwoven fabric, in contact with the wearer's skin, is embossed to form a concave-convex structure. Through the concave-convex structure, the area to contact with the wearer's skin is reduced effectively so as to lower the sticky feel or malposition.

However, this nonwoven fabric has recessed portions, and most nonwoven fabrics use hydrophile fibers as the raw material. When a liquid reaches the surface of the absorbent products, hydrophile fibers are beneficial for rapid wetting and permeation. Because the surface energy of the hydrophile fibers is high, the liquid is absorbed around the fibers and the residues of the liquid remain between the fibers, particularly, between the raised portions and recessed portions. The liquid remains at the bottom of the recessed portions more easily, which results in that the user may feel humid, sticky and wet. Especially, when this hydrophile nonwoven fabric is used to the surface of a baby diaper, it is easy to cause skin redness, eczema and other allergies due to poor dry performance. Besides, because the concave-convex portions are pressed by external forces, it is difficult to maintain an obvious lumpy effect and the area to contact with the wearer's skin is not reduced.

### SUMMARY OF THE INVENTION

The primary object of the present invention is to overcome the drawbacks of the prior art and provide a three-dimensional nonwoven fabric with a pore structure. The three-dimensional nonwoven fabric has an obvious lumpy effect and quickens liquid permeation and reduces residues. The three-dimensional nonwoven fabric has a high permeating speed and is dry and comfortable.

In order to achieve the aforesaid object, a three-dimensional nonwoven fabric with a pore structure is provided. The three-dimensional nonwoven fabric has raised regions, recessed regions, and the pore structure. The raised regions and the recessed regions are defined on the basis of a nonwoven application surface. Fiber densities from the raised regions to the recessed regions are gradient. The fiber density of the edges transiting from the raised regions to the recessed regions is higher than that of the tops of the raised regions. The fiber density of the edges is 1.5 to 30 times of the fiber density of the tops of the raised regions. The fiber density of the recessed regions is 1.5 to 20 times of the fiber density of the tops of the raised regions. A height difference between the raised regions and the recessed regions is greater than 0.2 mm. The average area of each of pores of the pore structure is smaller than 20 mm2. The porosity is lower than 50%. The area of the raised regions accounts for 20 to 80% of the total area of the three-dimensional nonwoven fabric.

Preferably, the pore structure is located in the raised regions or/and the recessed regions.

Preferably, the recessed regions surround the raised regions.

Preferably, the raised regions surround the recessed regions.

Preferably, the height difference between the raised regions and the recessed regions is greater than 0.3 mm.

Preferably, the pores of the recessed regions having the pore structure extend along the raised regions toward the recessed regions having the pore structure.

Preferably, the pores of the recessed regions having the pore structure extend along the recessed regions toward the raised regions.

Preferably, the three-dimensional nonwoven fabric is composed of a surface layer and at least one bottom layer. The surface layer is the surface of the raised regions. The surface layer is a hydrophobic surface layer.

Preferably, the pores of the raised regions having the pore structure extend from the surface of the raised regions toward the underside of the raised regions.

Preferably, the pores of the raised regions having the pore structure extend from the underside of the raised regions toward the surface of the raised regions.

Preferably, the pores are funnel-shaped pores.

The beneficial effects of the present invention are as follows. The three-dimensional nonwoven fabric has raised regions, recessed regions and a pore structure. Fiber densities from the raised regions to the recessed regions are gradient. The raised regions in contact with the skin have a low fiber density to bring a soft and comfortable feel to the user, and the edge regions have a strong compression capability, and the three-dimensional nonwoven fabric has a three-dimensional visual effect. The three-dimensional nonwoven fabric is composed of a surface layer and at least one bottom layer. The raised regions and the recessed regions have poles extending from the surface layer toward the bottom layer or extending from the bottom layer toward the surface layer. The surface layer is a hydrophobic surface layer. When the liquid reaches the raised regions, because the surface energy of the raised areas is low, the liquid flows from the edges of the raised regions to the pores, providing a guide function for the liquid to pass through the pores quickly so as to improve the dry performance of absorbent products. The liquid can be absorbed quickly. After the liquid is absorbed, the surface layer of the raised regions is a hydrophobic layer able to block the liquid from flowing back to the surface of the nonwoven fabric through capillary action, achieving a dry and comfortable effect. The height difference between the raised regions and the poles is greater than 0.2 mm. Even the liquid flows back to a portion of the surface, the user's skin is in contact with the raised regions only. The user will not feel moist. Besides, the three-dimensional nonwoven fabric has different fiber densities and fiber viscosity nodes to maintain the raised structure and the pore structure more strongly.

### BRIEF DESCRIPTION OF THE DRAWINGS

FIG. 1 is a top view of a three-dimensional nonwoven fabric in accordance with a first embodiment of the present invention;

FIG. 1A is a sectional view taken along line P-P of FIG. 1;

FIG. 1B is a sectional view taken along line Q-Q of FIG. 1;

FIG. 1C is a sectional view taken along line S-S of FIG. 1;

FIG. 1D is a schematic view showing the processing of the three-dimensional nonwoven fabric of the present invention;

FIG. 1E is a schematic view showing the processing equipment of the three-dimensional nonwoven fabric of the present invention;

FIG. 2 is a sectional view of a second embodiment of the present invention;

FIG. 3 is a top view of a three-dimensional nonwoven fabric in accordance with a third embodiment of the present invention;

FIG. 4 is a top view of a three-dimensional nonwoven fabric in accordance with a fourth embodiment of the present invention;

FIG. 4A is a sectional view taken along line P-P of FIG. 4;

FIG. 4B is a sectional view taken along line Q-Q of FIG. 4;

FIG. 5 is a top view of a three-dimensional nonwoven fabric in accordance with a fifth embodiment of the present invention;

FIG. 5A is a sectional view taken along line P-P of FIG. 5;

FIG. 6 is a top view of a three-dimensional nonwoven fabric in accordance with a sixth embodiment of the present invention;

FIG. 6A is a sectional view taken along line P-P of FIG. 6;

FIG. 6B is a sectional view taken along line Q-Q of FIG. 6;

FIG. 6C is a sectional view taken along line S-S of FIG. 6;

FIG. 7 is a top view of a three-dimensional nonwoven fabric in accordance with a seventh embodiment of the present invention;

FIG. 7A is a sectional view taken along line P-P of FIG. 7;

FIG. 7B is a sectional view taken along line Q-Q of FIG. 7;

FIG. 7C is a sectional view taken along line S-S of FIG. 7;

FIG. 8 is a top view of a three-dimensional nonwoven fabric in accordance with an eighth embodiment of the present invention;

FIG. 8A is a sectional view taken along line P-P of FIG. 8;

FIG. 8B is a sectional view taken along line Q-Q of FIG. 8;

FIG. 9 is a top view of a three-dimensional nonwoven fabric in accordance with a ninth embodiment of the present invention;

FIG. 9A is a sectional view taken along line P-P of FIG. 9;

FIG. 9B is a sectional view taken along line Q-Q of FIG. 9;

FIG. 10 is a top view of a three-dimensional nonwoven fabric in accordance with a tenth embodiment of the present invention;

FIG. 10A is a sectional view taken along line P-P of FIG. 10;

FIG. 11 is a top view of a three-dimensional nonwoven fabric in accordance with an eleventh embodiment of the present invention;

FIG. 11A is a sectional view taken along line P-P of FIG. 11;

FIG. 12 is a top view of a three-dimensional nonwoven fabric in accordance with a twelfth embodiment of the present invention;

FIG. 12A is a sectional view taken along line P-P of FIG. 12;

FIG. 13 is a top view of a three-dimensional nonwoven fabric in accordance with a thirteenth embodiment of the present invention;

FIG. 13A is a sectional view taken along line P-P of FIG. 13;

FIG. 14 is a top view of a three-dimensional nonwoven fabric in accordance with a fourteenth embodiment of the present invention;

FIG. 14A is a sectional view taken along line P-P of FIG. 14;

FIG. 15 is a top view of a three-dimensional nonwoven fabric in accordance with a fifteenth embodiment of the present invention;

FIG. 15A is a sectional view taken along line P-P of FIG. 15;

FIG. 16 is a top view of a three-dimensional nonwoven fabric in accordance with a sixteenth embodiment of the present invention;

FIG. 16A is a sectional view taken along line P-P of FIG. 16;

FIG. 17 is a top view of a three-dimensional nonwoven fabric in accordance with a seventeenth embodiment of the present invention; and

FIG. 17A is a sectional view taken along line P-P of FIG. 17.

### DETAILED DESCRIPTION OF THE PREFERRED EMBODIMENTS

Embodiments of the present invention will now be described, by way of example only, with reference to the accompanying drawings.

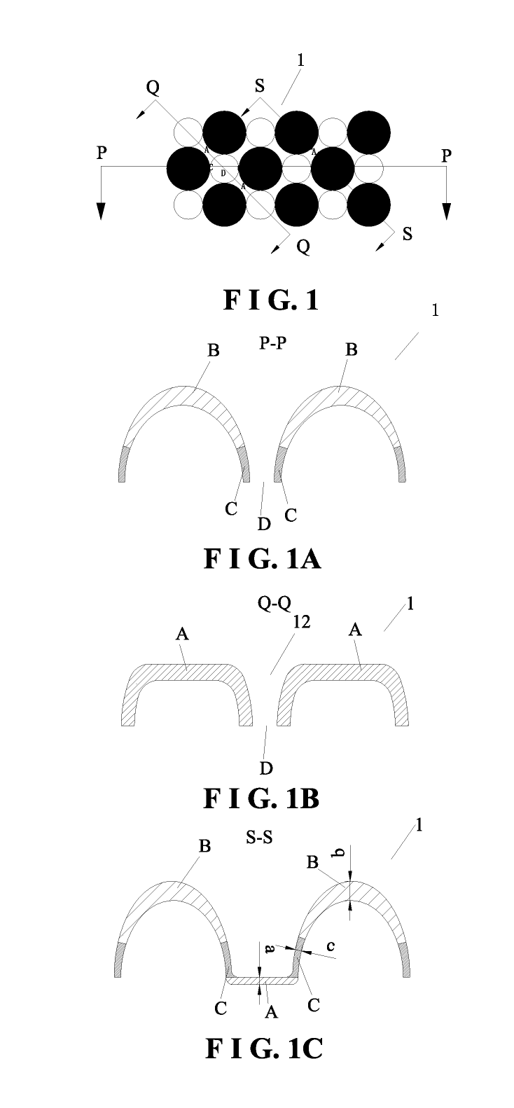

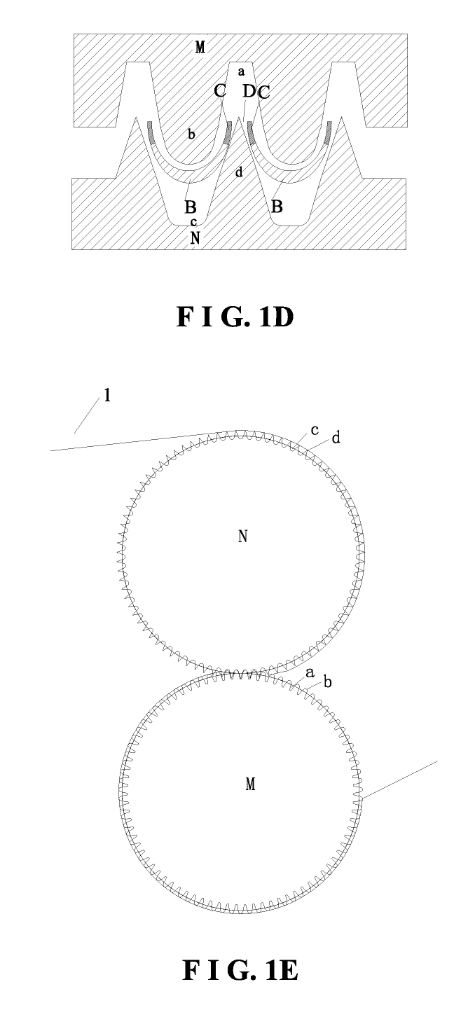

As shown in FIG. 1 through FIG. 17A, the present invention discloses a three-dimensional nonwoven fabric with a pore structure. The three-dimensional nonwoven fabric has raised regions B, recessed regions A, and the pore structure. The raised regions B and the recessed regions A are defined on the basis of a nonwoven application surface. Fiber densities from the raised regions B to the recessed regions A are gradient, that is, the fiber density of the edges transiting from the raised regions B to the recessed regions A is higher than that of the tops of the raised regions B, and the fiber density of the edges is 1.5 to 30 times of the fiber density of the tops of the raised regions B; the fiber density of the recessed regions A is 1.5 to 20 times of the fiber density of the tops of the raised regions B; a height difference between the raised regions B and the recessed regions A is greater than 0.2 mm; the average area of each single pore of the pore structure is smaller than 20 mm2, and the porosity is lower than 50%; and the area of the raised regions B accounts for 20 to 80% of the total area of the three-dimensional nonwoven fabric.

### First Embodiment

FIG. 1, FIG. 1A, FIG. 1B, and FIG. 1C illustrate a three-dimensional nonwoven fabric 1 having raised regions and recessed regions with a pore structure. The three-dimensional nonwoven fabric 1 has raised regions B and recessed regions A to be in contact with the user's skin. Seen from the top view, the raised regions B are circular, each like an “island” disposed in the recessed regions A. From the raised regions B to the recessed regions A is uniformly transited. Fiber densities from the raised regions B to the recessed regions A are gradient. The edges transiting from the raised regions B to the recessed regions A, close to the recessed regions A, are defined as C regions. The fiber density of the C regions is 1.5 to 30 times of the fiber density of the raised regions B. Preferably, the fiber density of the C regions is 1.5 to 5 times of the fiber density of the raised regions B. The fiber density of the recessed regions A is 1.5 to 20 times of the fiber density of the raised regions B. Preferably, the fiber density of the recessed regions A is 1.5 to 3 times of the fiber density of the raised regions B. In this way, the raised regions in contact with the skin have a soft and comfortable feel to the user, and the edge regions have a strong compression capability, and the three-dimensional nonwoven fabric has a three-dimensional visual effect. The recessed regions A have a pore structure D. The area of the D region is smaller than 20 mm2 Preferably, the area of the D region is smaller than 5 mm2 The porosity is lower than 50%. Preferably, the porosity is lower than 30%. The pore structure has a geometric shape, such as a circle, a square, a rectangle or a combination thereof. When a liquid flows from the edges of the raised areas B to the pores, the pore structure D provides a guide function for the liquid to pass through the pores quickly so as to improve the dry performance of absorbent products. The liquid can be absorbed quickly. A height difference between the raised regions B and the recessed regions A is greater than 0.2 mm. Preferably, the height difference is greater than 0.3 mm.

Test Method of Fiber Density Ratio:

The three-dimensional nonwoven fabric 1 with the pore structure is formed by a special processing on a flat nonwoven fabric. It can be considered the number of the fibers throughout the nonwoven fabric is the same before the formation of the three-dimensional nonwoven fabric 1. After the formation of the three-dimensional nonwoven fabric 1, the fiber densities of the raised regions B, the recessed regions A, and the edge regions C have a change.

By using the model XTL-200 electron microscope to measure the thickness of the nonwoven fabric, the thickness of the raised region B of the nonwoven fabric is b, the thickness of the recessed region A of the nonwoven fabric is a, and the thickness of the edge region C of the nonwoven fabric is c. Through the following formula, the fiber density ratio of each region is as follows:

The number of the fibers throughout the nonwoven fabric is set as X before the formation of the three-dimensional nonwoven fabric 1.

The fiber density ratio of the edge region to the raised region is

$$\frac{X \text{/} c}{X \text{/} b} = \frac{b}{c}$$

The fiber density ratio of the recessed region to the raised region is

$$\frac{X \text{/} a}{X \text{/} b} = \frac{b}{a}$$

Test Method of Porosity:

As shown in FIG. 1, by using the model XTL-200 electron microscope, the total area of the analyzed region is in, and the sum of the area of the pore region D within the analyzed region is ml.

The porosity is

$$\frac{ml}{m} \times 100 \%$$

As shown in FIG. 1D and FIG. 1E, a common nonwoven fabric is processed by a pair of concave and convex rollers M, N to mesh with each other at a speed of 5-100 meter/minute. The temperature of the concave roller M is set in the range of 40-200° C. The temperature of the convex roller N is set in the range of 30-200° C. The concave roller M has protrusions b and cavities a around the protrusions b. The height of each protrusion b is in the range of 0.5-45 mm. The depth of each cavity a is 0.5-50 mm. The convex roller N has cavities c and needles d thereon. The depth of each cavity c is 0.5-50 mm. The height of each needle d is in the range of 0.5-45 mm. The gap for the concave roller M and the convex roller N to mesh with each other can be adjusted. The gap is in the range of 0.1-45 mm. When the concave roller and the convex roller are in mesh, the nonwoven fabric is pressed by the protrusions b of the concave roller M and the cavities c of the convex roller N and heated to form the raised regions B. The protrusions b are inserted into the cavities C. The gap between the bottom of the protrusion b and the opening of the cavity c is the least. Under the action of temperature and pressure, the nonwoven fabric is formed with high-density regions A, C. The gap from the bottom to the top of the protrusion b is gradually increased. The depth of the cavity c is much larger than the height of the projection b, so that the protrusion b is not in contact with the bottom of the cavity c. The nonwoven fabric won't be compressed to form the low-density regions B. The nonwoven fabric is pierced by the cavities a of the concave roller M and the needles d of the convex roller N and heated to form the pore structure D. The concave roller M and the convex roller N mesh with each other and are driven by a transmission mechanism. The concave roller M and the convex roller N are heating rollers. The heating temperature and the gap between the concave roller M and the convex roller N to mesh with each other can be set.

### Second Embodiment

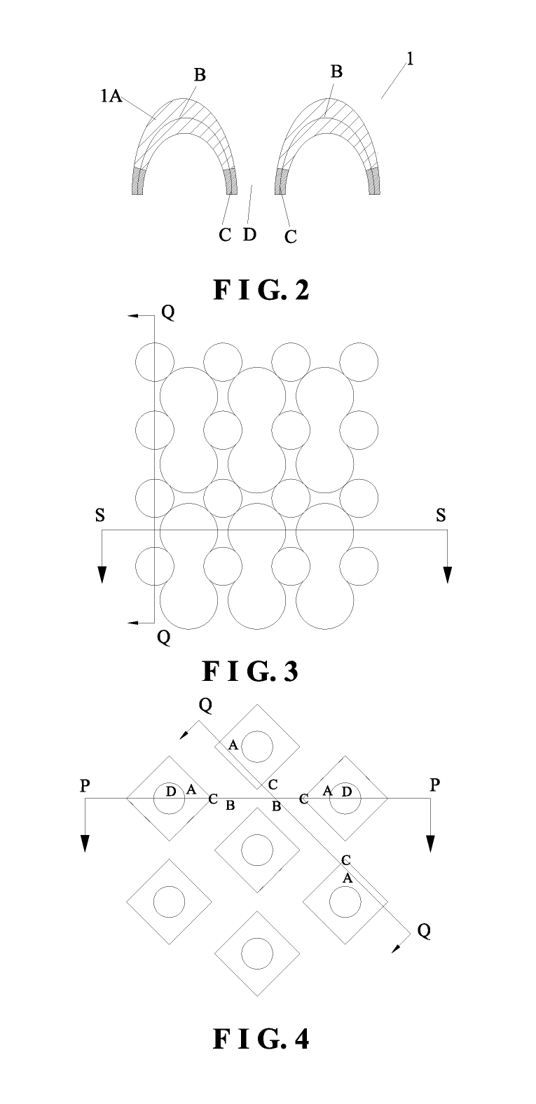

FIG. 2 illustrates a three-dimensional nonwoven fabric 1 having raised regions and recessed regions with a pore structure. The three-dimensional nonwoven fabric 1 has a surface layer 1A and a bottom layer 1B. The surface layer 1A is a hydrophobic layer and has the raised regions B and recessed regions A to be in contact with the user's skin. Seen from the top view, the raised regions B are circular, each like an “island” disposed in the recessed regions A. From the raised regions B to the recessed regions A is uniformly transited. Fiber densities from the raised regions B to the recessed regions A are gradient. The edges transiting from the raised regions B to the recessed regions A, close to the recessed regions A, are defined as C regions. The fiber density of the C regions is 1.5 to 30 times of the fiber density of the raised regions B. Preferably, the fiber density of the C regions is 1.5 to 5 times of the fiber density of the raised regions B. The fiber density of the recessed regions A is 1.5 to 20 times of the fiber density of the raised regions B. Preferably, the fiber density of the recessed regions A is 1.5 to 3 times of the fiber density of the raised regions B. The recessed regions A have a pore structure D. The area of the D region is smaller than 20 mm2 Preferably, the area of the D region is smaller than 5 mm2 The porosity is lower than 50%. Preferably, the porosity is lower than 30%. A height difference between the raised regions B and the recessed regions A is greater than 0.2 mm. Preferably, the height difference is greater than 0.3 mm. When the hydrophobic surface layer 1A enables the liquid to reach the raised regions B, because the surface energy of the raised areas A is low, the liquid flows from the edges of the raised regions B to the pores, providing a guide function for the liquid to pass through the pores quickly so as to improve the dry performance of absorbent products. The liquid can be absorbed quickly. After the liquid is absorbed, the surface layer 1A of the raised regions 1B is a hydrophobic layer able to block the liquid from flowing back to the surface of the nonwoven fabric through capillary action, achieving a dry and comfortable effect.

### Third Embodiment

FIG. 3 illustrates a three-dimensional nonwoven fabric 1 having raised regions and recessed regions with a pore structure. The three-dimensional nonwoven fabric 1 has raised regions B and recessed regions A to be in contact with the user's skin. Seen from the top view, the raised regions B each have a calabash shape, like an “island” disposed in the recessed regions A. From the raised regions B to the recessed regions A is uniformly transited. Fiber densities from the raised regions B to the recessed regions A are gradient. The edges transiting from the raised regions B to the recessed regions A, close to the recessed regions A, are defined as C regions. The fiber density of the C regions is 1.5 to 30 times of the fiber density of the raised regions B. Preferably, the fiber density of the C regions is 2 to 5 times of the fiber density of the raised regions B. The fiber density of the recessed regions A is 1.5 to 20 times of the fiber density of the raised regions B. Preferably, the fiber density of the recessed regions A is 1.5 to 3 times of the fiber density of the raised regions B. The recessed regions A have a pore structure D. The area of the D region is smaller than 20 mm2 Preferably, the area of the D region is smaller than 5 mm2 The porosity is lower than 50%. Preferably, the porosity is lower than 30%. A height difference between the raised regions B and the recessed regions A is greater than 0.2 mm. Preferably, the height difference is greater than 0.3 mm. The raised regions have a geometric shape, such as a circle, a square, a rectangle or a combination thereof.

### Fourth Embodiment

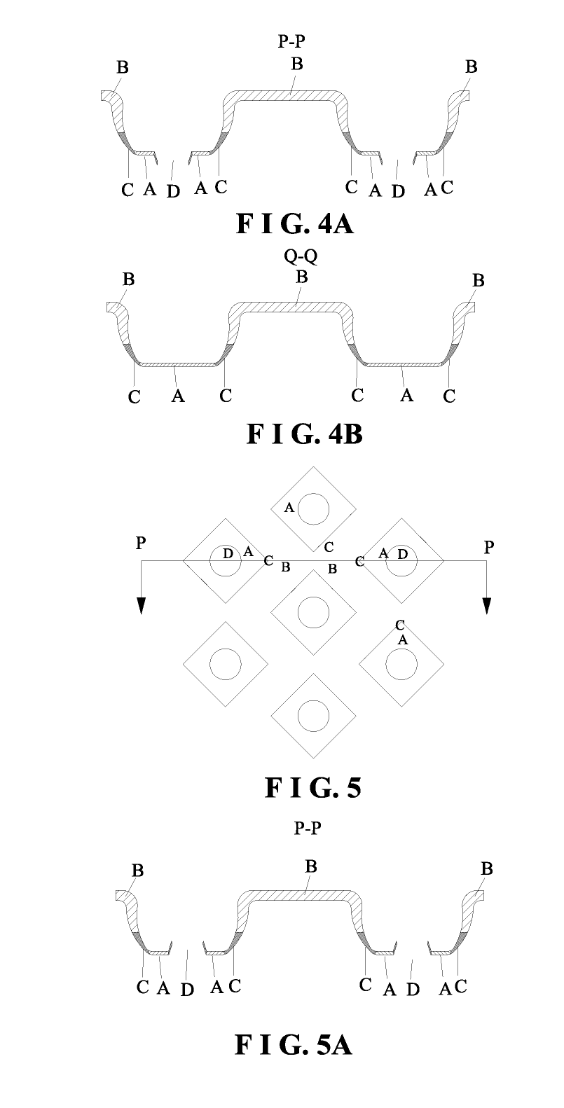

FIG. 4, FIG. 4A, and FIG. 4B illustrate a three-dimensional nonwoven fabric 1 having raised regions and recessed regions with a pore structure. The three-dimensional nonwoven fabric 1 has raised regions B and recessed regions A to be in contact with the user's skin. The raised regions B are continuous planes, like the “sea” surrounding the recessed regions A. From the raised regions B to the recessed regions A is uniformly transited. Fiber densities from the raised regions B to the recessed regions A are gradient. The edges transiting from the raised regions B to the recessed regions A, close to the recessed regions A, are defined as C regions. The fiber density of the C regions is 1.5 to 30 times of the fiber density of the raised regions B. Preferably, the fiber density of the C regions is 1.5 to 5 times of the fiber density of the raised regions B. The fiber density of the recessed regions A is 1.5 to 20 times of the fiber density of the raised regions B. Preferably, the fiber density of the recessed regions A is 1.5 to 3 times of the fiber density of the raised regions B. The recessed regions A have a pore structure D. The pore structure D extends from the surface of the recessed regions toward the underside of the recessed regions. The area of the D region is smaller than 20 mm2 Preferably, the area of the D region is smaller than 5 mm2 The porosity is lower than 50%. Preferably, the porosity is lower than 30%. A height difference between the raised regions B and the recessed regions A is greater than 0.2 mm. Preferably, the height difference is greater than 0.3 mm. The recessed regions have a geometric shape, such as a circle, a square, a rectangle or a combination thereof.

### Fifth Embodiment

FIG. 5 and FIG. 5A illustrate a three-dimensional nonwoven fabric 1 having raised regions and recessed regions with a pore structure. The three-dimensional nonwoven fabric 1 has raised regions B and recessed regions A to be in contact with the user's skin. The raised regions B are continuous planes, like the “sea” surrounding the recessed regions A. From the raised regions B to the recessed regions A is uniformly transited. Fiber densities from the raised regions B to the recessed regions A are gradient. The edges transiting from the raised regions B to the recessed regions A, close to the recessed regions A, are defined as C regions. The fiber density of the C regions is 1.5 to 30 times of the fiber density of the raised regions B. Preferably, the fiber density of the C regions is 1.5 to 5 times of the fiber density of the raised regions B. The fiber density of the recessed regions A is 1.5 to 20 times of the fiber density of the raised regions B. Preferably, the fiber density of the recessed regions A is 1.5 to 3 times of the fiber density of the raised regions B. The recessed regions A have a pore structure D. The pore structure D extends from the underside of the recessed regions toward the surface of the recessed regions. The area of the D region is smaller than 20 mm2 Preferably, the area of the D region is smaller than 5 mm2 The porosity is lower than 50%. Preferably, the porosity is lower than 30%. A height difference between the raised regions B and the recessed regions A is greater than 0.2 mm. Preferably, the height difference is greater than 0.3 mm.

### Sixth Embodiment

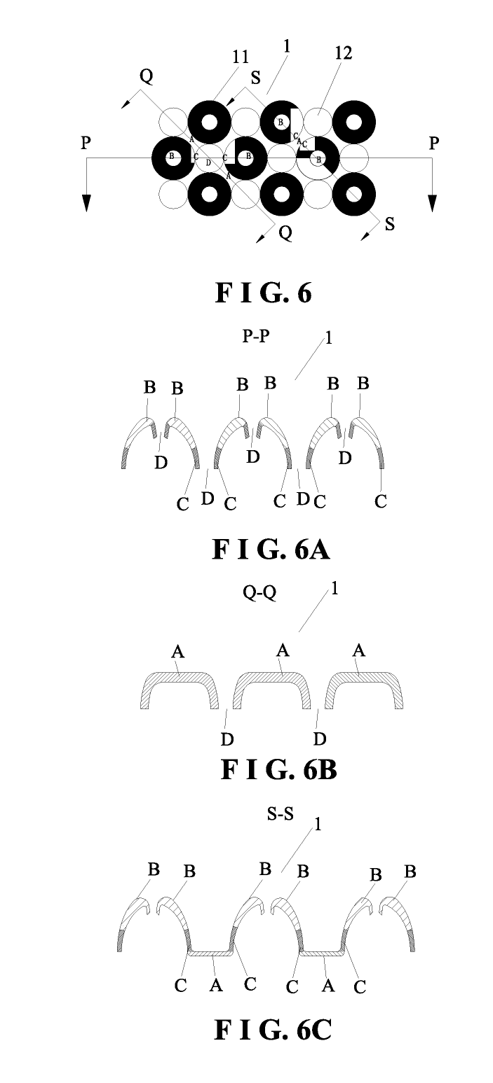

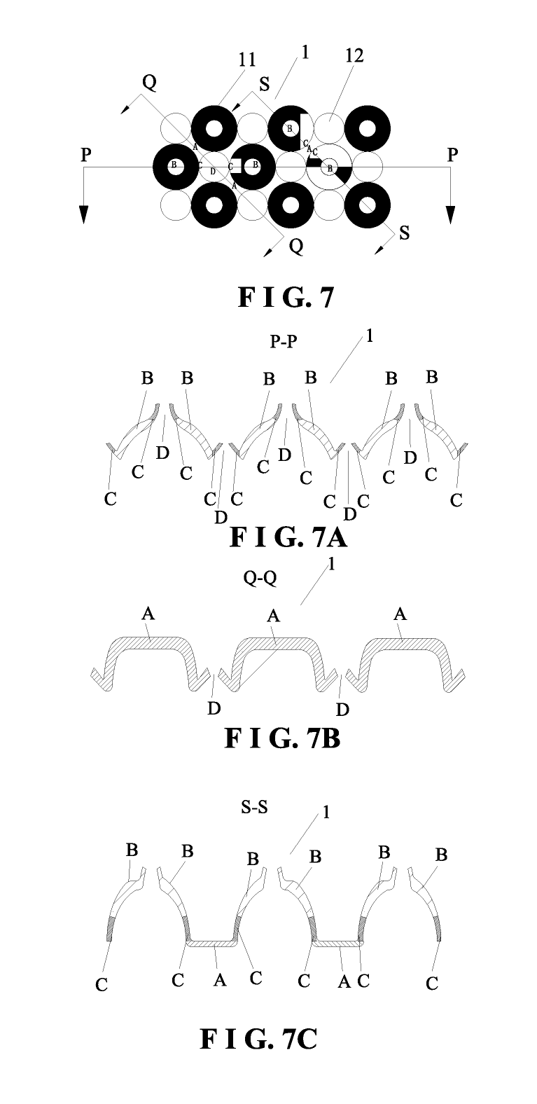

FIG. 6, FIG. 6A, FIG. 6B, and FIG. 6C illustrate a three-dimensional nonwoven fabric 1 having raised regions and recessed regions with a pore structure. The three-dimensional nonwoven fabric 1 has raised regions B and recessed regions A to be in contact with the user's skin. The raised regions B are each like an “island” disposed in the recessed regions A. From the raised regions B to the recessed regions A is uniformly transited. Fiber densities from the raised regions B to the recessed regions A are gradient. The edges transiting from the raised regions B to the recessed regions A, close to the recessed regions A, are defined as C regions. The fiber density of the C regions is 1.5 to 30 times of the fiber density of the raised regions B. Preferably, the fiber density of the C regions is 1.5 to 5 times of the fiber density of the raised regions B. The fiber density of the recessed regions A is 1.5 to 20 times of the fiber density of the raised regions B. Preferably, the fiber density of the recessed regions A is 1.5 to 3 times of the fiber density of the raised regions B. The raised regions B and the recessed regions A each have a pore structure D. The pore structure D extends from the surface of the raised regions B and the recessed regions A toward the underside of the raised regions B and the recessed regions A. The area of the D region is smaller than 20 mm2 Preferably, the area of the D region is smaller than 5 mm2 The porosity is lower than 50%. Preferably, the porosity is lower than 30%. A height difference between the raised regions B and the recessed regions A is greater than 0.2 mm. Preferably, the height difference is greater than 0.3 mm.

### Seventh Embodiment

FIG. 7, FIG. 7A, FIG. 7B, and FIG. 7C illustrate a three-dimensional nonwoven fabric 1 having raised regions and recessed regions with a pore structure. The three-dimensional nonwoven fabric 1 has raised regions B and recessed regions A to be in contact with the user's skin. The raised regions B are each like an “island” disposed in the recessed regions A. From the raised regions B to the recessed regions A is uniformly transited. Fiber densities from the raised regions B to the recessed regions A are gradient. The edges transiting from the raised regions B to the recessed regions A, close to the recessed regions A, are defined as C regions. The fiber density of the C regions is 1.5 to 30 times of the fiber density of the raised regions B. Preferably, the fiber density of the C regions is 1.5 to 5 times of the fiber density of the raised regions B. The fiber density of the recessed regions A is 1.5 to 20 times of the fiber density of the raised regions B. Preferably, the fiber density of the recessed regions A is 1.5 to 3 times of the fiber density of the raised regions B. The raised regions B and the recessed regions A each have a pore structure D. The pore structure D extends from the underside of the raised regions B and the recessed regions A toward the surface of the raised regions B and the recessed regions A. The area of the D region is smaller than 20 mm2 Preferably, the area of the D region is smaller than 5 mm2 The porosity is lower than 50%. Preferably, the porosity is lower than 30%. A height difference between the raised regions B and the recessed regions A is greater than 0.2 mm. Preferably, the height difference is greater than 0.3 mm.

### Eighth Embodiment

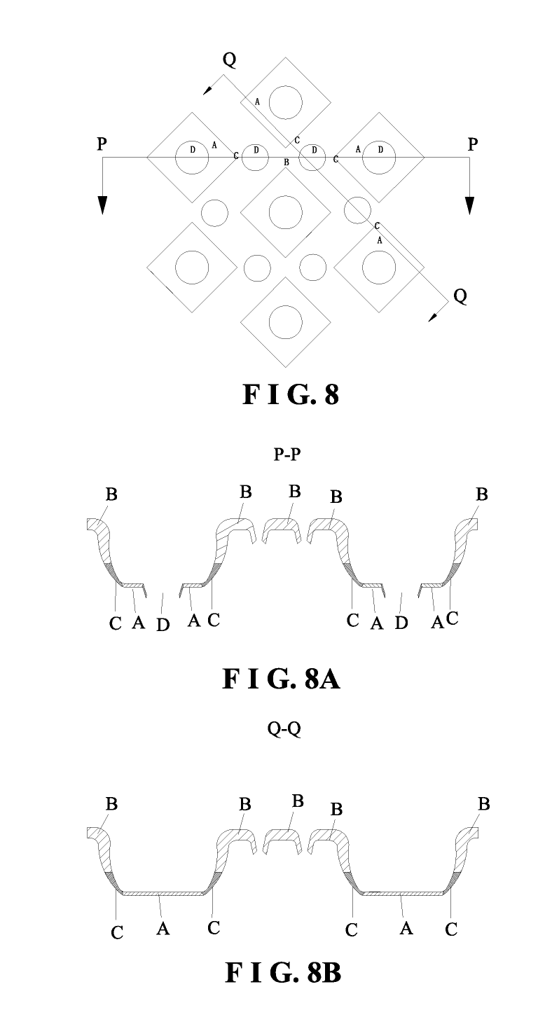

FIG. 8, FIG. 8A and FIG. 8B illustrate a three-dimensional nonwoven fabric 1 having raised regions and recessed regions with a pore structure. The three-dimensional nonwoven fabric 1 has raised regions B and recessed regions A to be in contact with the user's skin. The raised regions B are like the “sea” surrounding the recessed regions A. From the raised regions B to the recessed regions A is uniformly transited. Fiber densities from the raised regions B to the recessed regions A are gradient. The edges transiting from the raised regions B to the recessed regions A, close to the recessed regions A, are defined as C regions. The fiber density of the C regions is 1.5 to 30 times of the fiber density of the raised regions B. Preferably, the fiber density of the C regions is 1.5 to 5 times of the fiber density of the raised regions B. The fiber density of the recessed regions A is 1.5 to 20 times of the fiber density of the raised regions B. Preferably, the fiber density of the recessed regions A is 1.5 to 3 times of the fiber density of the raised regions B. The raised regions B and the recessed regions A each have a pore structure D. The pore structure D extends from the surface of the raised regions B and the recessed regions A toward the underside of the raised regions B and the recessed regions A. The area of the D region is smaller than 20 mm2 Preferably, the area of the D region is smaller than 5 mm2 The porosity is lower than 50%. Preferably, the porosity is lower than 30%. A height difference between the raised regions B and the recessed regions A is greater than 0.2 mm. Preferably, the height difference is greater than 0.3 mm.

### Ninth Embodiment

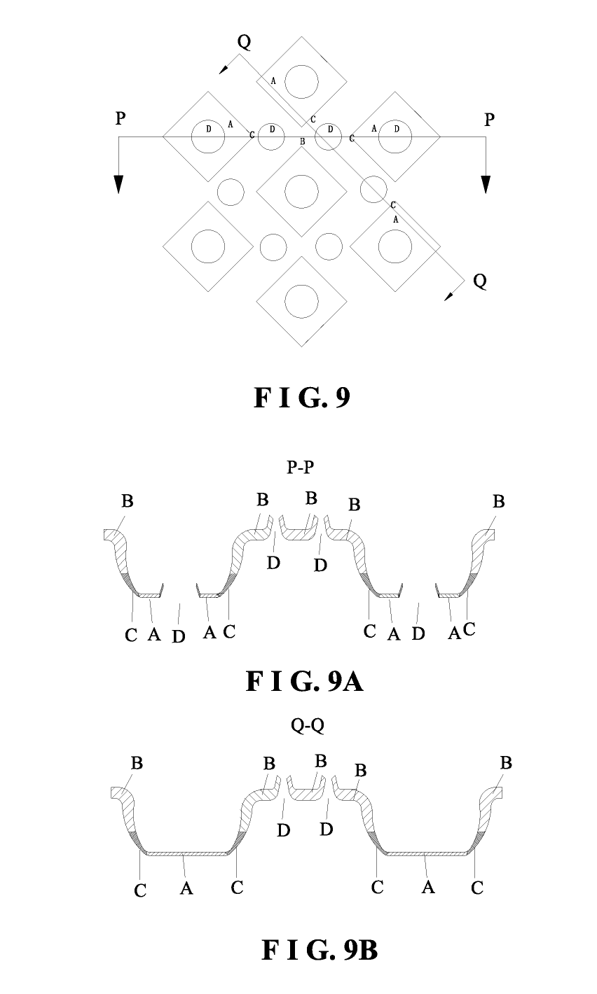

FIG. 9, FIG. 9A and FIG. 9B illustrate a three-dimensional nonwoven fabric 1 having raised regions and recessed regions with a pore structure. The three-dimensional nonwoven fabric 1 has raised regions B and recessed regions A to be in contact with the user's skin. The raised regions B are like the “sea” surrounding the recessed regions A. From the raised regions B to the recessed regions A is uniformly transited. Fiber densities from the raised regions B to the recessed regions A are gradient. The edges transiting from the raised regions B to the recessed regions A, close to the recessed regions A, are defined as C regions. The fiber density of the C regions is 1.5 to 30 times of the fiber density of the raised regions B. Preferably, the fiber density of the C regions is 1.5 to 5 times of the fiber density of the raised regions B. The fiber density of the recessed regions A is 1.5 to 20 times of the fiber density of the raised regions B. Preferably, the fiber density of the recessed regions A is 1.5 to 3 times of the fiber density of the raised regions B. The raised regions B and the recessed regions A each have a pore structure D. The pore structure D extends from the underside of the raised regions B and the recessed regions A toward the surface of the raised regions B and the recessed regions A. The area of the D region is smaller than 20 mm2 Preferably, the area of the D region is smaller than 5 mm2 The porosity is lower than 50%. Preferably, the porosity is lower than 30%. A height difference between the raised regions B and the recessed regions A is greater than 0.2 mm. Preferably, the height difference is greater than 0.3 mm.

### Tenth Embodiment

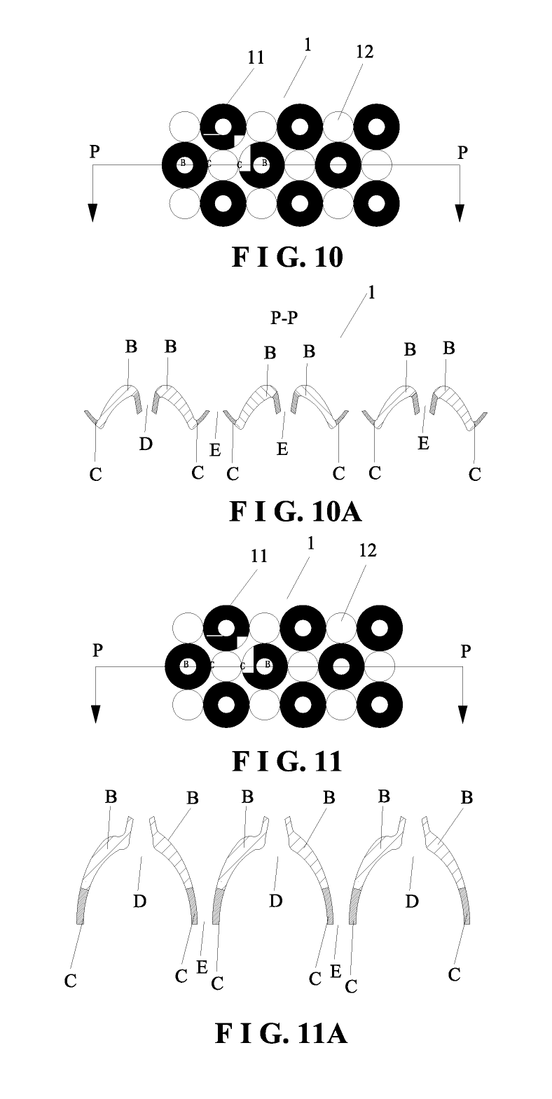

FIG. 10 and FIG. 10A illustrate a three-dimensional nonwoven fabric 1 having raised regions and recessed regions with a pore structure. The three-dimensional nonwoven fabric 1 has raised regions B and recessed regions A to be in contact with the user's skin. The raised regions B are each like an “island” disposed in the recessed regions A. From the raised regions B to the recessed regions A is uniformly transited. Fiber densities from the raised regions B to the recessed regions A are gradient. The edges transiting from the raised regions B to the recessed regions A, close to the recessed regions A, are defined as C regions. The fiber density of the C regions is 1.5 to 30 times of the fiber density of the raised regions B. Preferably, the fiber density of the C regions is 1.5 to 5 times of the fiber density of the raised regions B. The fiber density of the recessed regions A is 1.5 to 20 times of the fiber density of the raised regions B. Preferably, the fiber density of the recessed regions A is 1.5 to 3 times of the fiber density of the raised regions B. The raised regions B have a pore structure D. The pore structure D extends from the surface of the raised regions B toward the underside of the raised regions B. The recessed regions A have a pore structure E. The pore structure E extends from the underside of the recessed regions A toward the surface of the recessed regions A. The area of the D region is smaller than 20 mm2 Preferably, the area of the D region is smaller than 5 mm2 The porosity is lower than 50%. Preferably, the porosity is lower than 30%. A height difference between the raised regions B and the recessed regions A is greater than 0.2 mm. Preferably, the height difference is greater than 0.3 mm.

### Eleventh Embodiment

FIG. 11 and FIG. 11A illustrate a three-dimensional nonwoven fabric 1 having raised regions and recessed regions with a pore structure. The three-dimensional nonwoven fabric 1 has raised regions B and recessed regions A to be in contact with the user's skin. The raised regions B are each like an “island” disposed in the recessed regions A. From the raised regions B to the recessed regions A is uniformly transited. Fiber densities from the raised regions B to the recessed regions A are gradient. The edges transiting from the raised regions B to the recessed regions A, close to the recessed regions A, are defined as C regions. The fiber density of the C regions is 1.5 to 30 times of the fiber density of the raised regions B. Preferably, the fiber density of the C regions is 1.5 to 5 times of the fiber density of the raised regions B. The fiber density of the recessed regions A is 1.5 to 20 times of the fiber density of the raised regions B. Preferably, the fiber density of the recessed regions A is 1.5 to 3 times of the fiber density of the raised regions B. The raised regions B have a pore structure D. The pore structure D extends from the underside of the raised regions B toward the surface of the raised regions B. The recessed regions A have a pore structure E. The pore structure E extends from the surface of the recessed regions A toward the underside of the recessed regions A. The area of the D region is smaller than 20 mm2 Preferably, the area of the D region is smaller than 5 mm2 The porosity is lower than 50%. Preferably, the porosity is lower than 30%. A height difference between the raised regions B and the recessed regions A is greater than 0.2 mm. Preferably, the height difference is greater than 0.3 mm.

### Twelfth Embodiment

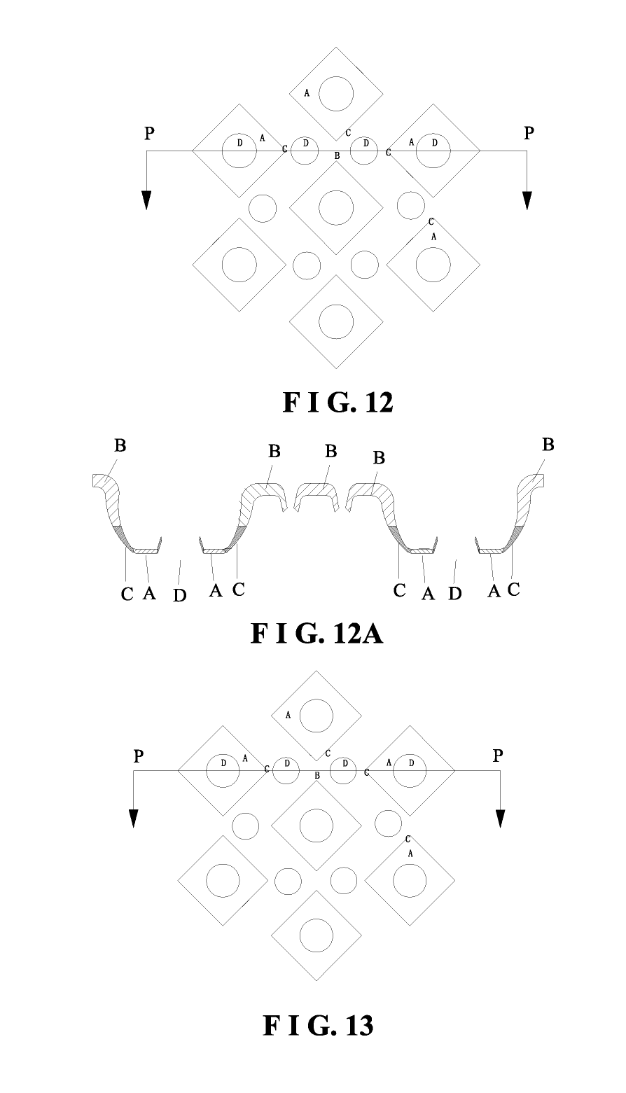

FIG. 12 and FIG. 12A illustrate a three-dimensional nonwoven fabric 1 having raised regions and recessed regions with a pore structure. The three-dimensional nonwoven fabric 1 has raised regions B and recessed regions A to be in contact with the user's skin. The raised regions B are like the “sea” surrounding the recessed regions A. From the raised regions B to the recessed regions A is uniformly transited. Fiber densities from the raised regions B to the recessed regions A are gradient. The edges transiting from the raised regions B to the recessed regions A, close to the recessed regions A, are defined as C regions. The fiber density of the C regions is 1.5 to 30 times of the fiber density of the raised regions B. Preferably, the fiber density of the C regions is 1.5 to 5 times of the fiber density of the raised regions B. The fiber density of the recessed regions A is 1.5 to 20 times of the fiber density of the raised regions B. Preferably, the fiber density of the recessed regions A is 1.5 to 3 times of the fiber density of the raised regions B. The raised regions B have a pore structure D. The pore structure D extends from the surface of the raised regions B toward the underside of the raised regions B. The recessed regions A have a pore structure E. The pore structure E extends from the underside of the recessed regions A toward the surface of the recessed regions A. The area of the D region is smaller than 20 mm2 Preferably, the area of the D region is smaller than 5 mm2 The porosity is lower than 50%. Preferably, the porosity is lower than 30%. A height difference between the raised regions B and the recessed regions A is greater than 0.2 mm. Preferably, the height difference is greater than 0.3 mm.

### Thirteenth Embodiment

FIG. 13 and FIG. 13A illustrate a three-dimensional nonwoven fabric 1 having raised regions and recessed regions with a pore structure. The three-dimensional nonwoven fabric 1 has raised regions B and recessed regions A to be in contact with the user's skin. The raised regions B are like the “sea” surrounding the recessed regions A. From the raised regions B to the recessed regions A is uniformly transited. Fiber densities from the raised regions B to the recessed regions A are gradient. The edges transiting from the raised regions B to the recessed regions A, close to the recessed regions A, are defined as C regions. The fiber density of the C regions is 1.5 to 30 times of the fiber density of the raised regions B. Preferably, the fiber density of the C regions is 1.5 to 5 times of the fiber density of the raised regions B. The fiber density of the recessed regions A is 1.5 to 20 times of the fiber density of the raised regions B. Preferably, the fiber density of the recessed regions A is 1.5 to 3 times of the fiber density of the raised regions B. The raised regions B have a pore structure D. The pore structure D extends from the underside of the raised regions B toward the surface of the raised regions B. The recessed regions A have a pore structure E. The pore structure E extends from the surface of the recessed regions A toward the underside of the recessed regions A. The area of the D region is smaller than 20 mm2 Preferably, the area of the D region is smaller than 5 mm2 The porosity is lower than 50%. Preferably, the porosity is lower than 30%. A height difference between the raised regions B and the recessed regions A is greater than 0.2 mm. Preferably, the height difference is greater than 0.3 mm.

### Fourteenth Embodiment

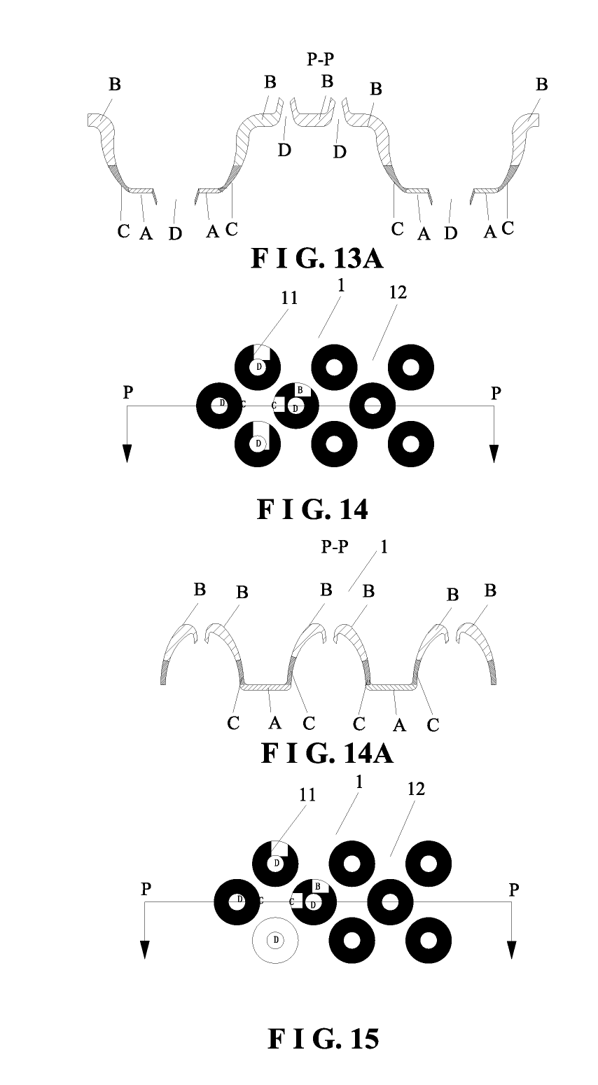

FIG. 14 and FIG. 14A illustrate a three-dimensional nonwoven fabric 1 having raised regions and recessed regions with a pore structure. The three-dimensional nonwoven fabric 1 has raised regions B and recessed regions A to be in contact with the user's skin. The raised regions B are each like an “island” disposed in the recessed regions A. From the raised regions B to the recessed regions A is uniformly transited. Fiber densities from the raised regions B to the recessed regions A are gradient. The edges transiting from the raised regions B to the recessed regions A, close to the recessed regions A, are defined as C regions. The fiber density of the C regions is 1.5 to 30 times of the fiber density of the raised regions B. Preferably, the fiber density of the C regions is 1.5 to 5 times of the fiber density of the raised regions B. The fiber density of the recessed regions A is 1.5 to 20 times of the fiber density of the raised regions B. Preferably, the fiber density of the recessed regions A is 1.5 to 3 times of the fiber density of the raised regions B. The raised regions B have a pore structure D. The pore structure D extends from the surface of the raised regions B toward the underside of the raised regions B. The recessed regions A don't have a pore structure. The area of the D region is smaller than 20 mm2 Preferably, the area of the D region is smaller than 5 mm2 The porosity is lower than 50%. Preferably, the porosity is lower than 30%. A height difference between the raised regions B and the recessed regions A is greater than 0.2 mm. Preferably, the height difference is greater than 0.3 mm.

### Fifteenth Embodiment

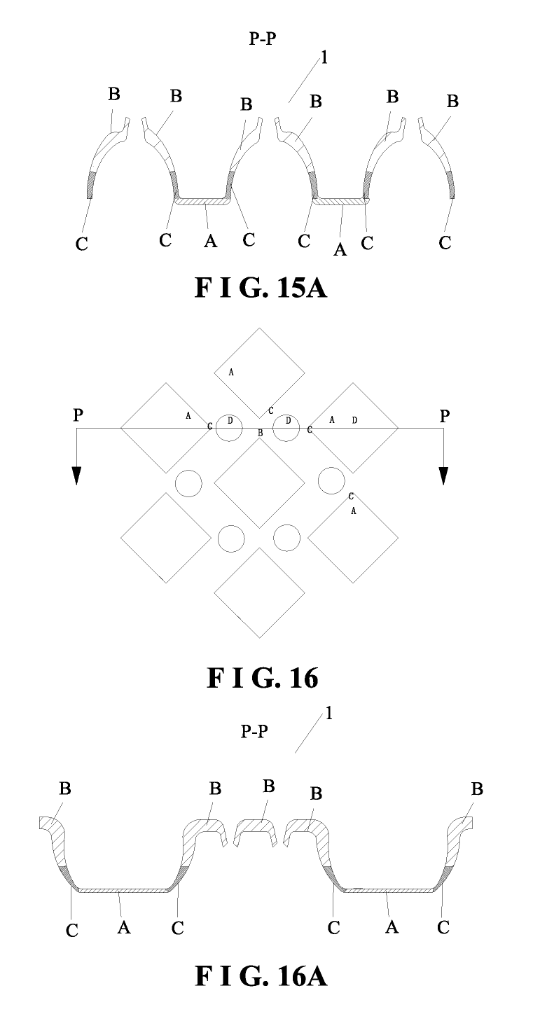

FIG. 15 and FIG. 15A illustrate a three-dimensional nonwoven fabric 1 having raised regions and recessed regions with a pore structure. The three-dimensional nonwoven fabric 1 has raised regions B and recessed regions A to be in contact with the user's skin. The raised regions B are each like an “island” disposed in the recessed regions A. From the raised regions B to the recessed regions A is uniformly transited. Fiber densities from the raised regions B to the recessed regions A are gradient. The edges transiting from the raised regions B to the recessed regions A, close to the recessed regions A, are defined as C regions. The fiber density of the C regions is 1.5 to 30 times of the fiber density of the raised regions B. Preferably, the fiber density of the C regions is 1.5 to 5 times of the fiber density of the raised regions B. The fiber density of the recessed regions A is 1.5 to 20 times of the fiber density of the raised regions B. Preferably, the fiber density of the recessed regions A is 1.5 to 3 times of the fiber density of the raised regions B. The raised regions B have a pore structure D. The pore structure D extends from the underside of the raised regions B toward the surface of the raised regions B. The recessed regions A don't have a pore structure. The area of the D region is smaller than 20 mm2 Preferably, the area of the D region is smaller than 5 mm2 The porosity is lower than 50%. Preferably, the porosity is lower than 30%. A height difference between the raised regions B and the recessed regions A is greater than 0.2 mm. Preferably, the height difference is greater than 0.3 mm.

### Sixteenth Embodiment

FIG. 16 and FIG. 16A illustrate a three-dimensional nonwoven fabric 1 having raised regions and recessed regions with a pore structure. The three-dimensional nonwoven fabric 1 has raised regions B and recessed regions A to be in contact with the user's skin. The raised regions B are like the “sea” surrounding the recessed regions A. From the raised regions B to the recessed regions A is uniformly transited. Fiber densities from the raised regions B to the recessed regions A are gradient. The edges transiting from the raised regions B to the recessed regions A, close to the recessed regions A, are defined as C regions. The fiber density of the C regions is 1.5 to 30 times of the fiber density of the raised regions B. Preferably, the fiber density of the C regions is 1.5 to 5 times of the fiber density of the raised regions B. The fiber density of the recessed regions A is 1.5 to 20 times of the fiber density of the raised regions B. Preferably, the fiber density of the recessed regions A is 1.5 to 3 times of the fiber density of the raised regions B. The raised regions B have a pore structure D. The pore structure D extends from the surface of the raised regions B toward the underside of the raised regions B. The recessed regions A don't have a pore structure. The area of the D region is smaller than 20 mm2 Preferably, the area of the D region is smaller than 5 mm2 The porosity is lower than 50%. Preferably, the porosity is lower than 30%. A height difference between the raised regions B and the recessed regions A is greater than 0.2 mm. Preferably, the height difference is greater than 0.3 mm.

### Seventeenth Embodiment

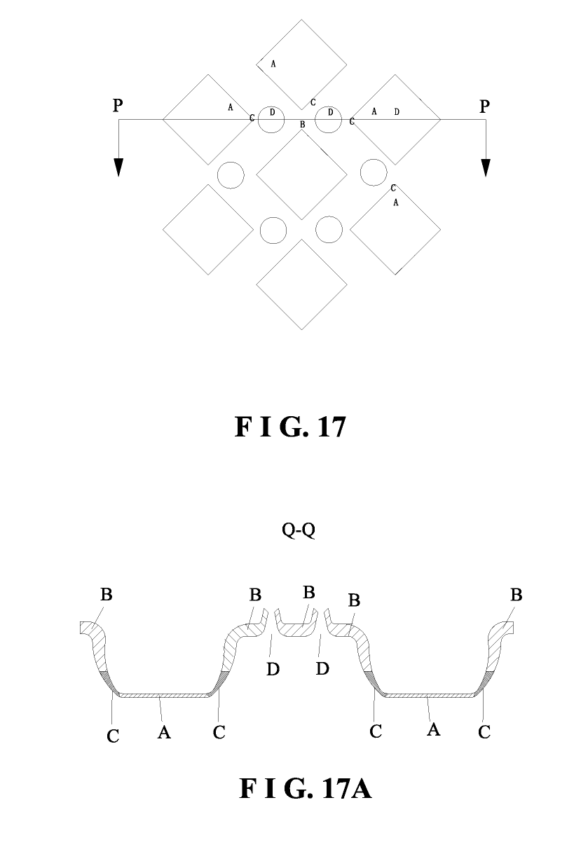

FIG. 17 and FIG. 17A illustrate a three-dimensional nonwoven fabric 1 having raised regions and recessed regions with a pore structure. The three-dimensional nonwoven fabric 1 has raised regions B and recessed regions A to be in contact with the user's skin. The raised regions B are like the “sea” surrounding the recessed regions A. From the raised regions B to the recessed regions A is uniformly transited. Fiber densities from the raised regions B to the recessed regions A are gradient. The edges transiting from the raised regions B to the recessed regions A, close to the recessed regions A, are defined as C regions. The fiber density of the C regions is 1.5 to 30 times of the fiber density of the raised regions B. Preferably, the fiber density of the C regions is 1.5 to 5 times of the fiber density of the raised regions B. The fiber density of the recessed regions A is 1.5 to 20 times of the fiber density of the raised regions B. Preferably, the fiber density of the recessed regions A is 1.5 to 3 times of the fiber density of the raised regions B. The raised regions B have a pore structure D. The pore structure D extends from the underside of the raised regions B toward the surface of the raised regions B. The recessed regions A don't have a pore structure. The area of the D region is smaller than 20 mm2 Preferably, the area of the D region is smaller than 5 mm2 The porosity is lower than 50%. Preferably, the porosity is lower than 30%. A height difference between the raised regions B and the recessed regions A is greater than 0.2 mm. Preferably, the height difference is greater than 0.3 mm.

Accordingly, the three-dimensional nonwoven fabric with a pore structure of the present invention has raised regions and recessed regions. The pore structure is disposed around the raised regions. The three-dimensional nonwoven fabric has different fiber densities. Pressure resistance performance of the high-density fiber regions enables the three-dimensional nonwoven fabric to have an obvious lumpy effect and quickens liquid permeation and reduces residues.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A three-dimensional nonwoven fabric with a pore structure, having raised regions, recessed regions, and the pore structure, the raised regions and the recessed regions being defined on the basis of a nonwoven application surface; characterized in that: fiber densities from the raised regions to the recessed regions are gradient, the fiber density of edges transiting from the raised regions to the recessed regions is higher than that of tops of the raised regions, the fiber density of the edges is 1.5 to 30 times of the fiber density of the tops of the raised regions; the fiber density of the recessed regions is 1.5 to 20 times of the fiber density of the tops of the raised regions; a height difference between the raised regions and the recessed regions is greater than 0.2 mm; an average area of each of pores of the pore structure is smaller than 20 mm2, porosity is lower than 50%; and the area of the raised regions accounts for 20 to 80% of the total area of the three-dimensional nonwoven fabric.

2. The three-dimensional nonwoven fabric with a pore structure as claimed in claim 1, wherein the pore structure is located in the raised regions or/and the recessed regions.

3. The three-dimensional nonwoven fabric with a pore structure as claimed in claim 1, wherein the recessed regions surround the raised regions.

4. The three-dimensional nonwoven fabric with a pore structure as claimed in claim 1, wherein the raised regions surround the recessed regions.

5. The three-dimensional nonwoven fabric with a pore structure as claimed in claim 1, wherein the height difference between the raised regions and the recessed regions is greater than 0.3 mm.

6. The three-dimensional nonwoven fabric with a pore structure as claimed in claim 2, wherein the pores of the recessed regions having the pore structure extend along the raised regions toward the recessed regions having the pore structure.

7. The three-dimensional nonwoven fabric with a pore structure as claimed in claim 2, wherein the pores of the recessed regions having the pore structure extend along the recessed regions toward the raised regions.

8. The three-dimensional nonwoven fabric with a pore structure as claimed in claim 1, wherein the three-dimensional nonwoven fabric is composed of a surface layer and at least one bottom layer, the surface layer is the surface of the raised regions, and the surface layer is a hydrophobic surface layer.

9. The three-dimensional nonwoven fabric with a pore structure as claimed in claim 2, wherein the pores of the raised regions having the pore structure extend from the surface of the raised regions toward the underside of the raised regions.

10. The three-dimensional nonwoven fabric with a pore structure as claimed in claim 2, wherein the pores of the raised regions having the pore structure extend from the underside of the raised regions toward the surface of the raised regions.

11. The three-dimensional nonwoven fabric with a pore structure as claimed in claim 1, wherein the pores are funnel-shaped pores.
